(19) United States
(12) Patent Application Publication
(10) Pub. No.: US 2020/0229953 A1
(43) Pub. Date: Jul. 23, 2020

(54) BIOABSORABLE FILAMENT MEDICAL DEVICES

(71) Applicant: W. L. Gore & Associates, Inc., Newark, DE (US)

(72) Inventors: Edward H. Cully, Newark, DE (US); Jeffrey B. Duncan, Flagstaff, AZ (US); Tom R. McDaniel, Flagstaff, AZ (US); David J. Messick, Flagstaff, AZ (US); Martin J. Sector, Phoenix, AZ (US)

(21) Appl. No.: 16/745,834

(22) Filed: Jan. 17, 2020

Related U.S. Application Data
(60) Provisional application No. 62/794,387, filed on Jan. 18, 2019.

(51) Int. Cl. A61F 2/90

(52) U.S. Cl. CPC A61F 2/90; A61F 2210/0004; A61F 2002/009; A61F 2250/0067; A61F 2240/001

(57) ABSTRACT

Various aspects of the present disclosure are directed toward apparatuses, systems, and methods that include a filament and a membrane arranged about the filament. The membrane may be configured to contain fragments of the filament and maintain structure of the membrane in response to the fracture or degradation of the filament.

### CROSS-REFERENCE TO RELATED APPLICATION

This application claims the benefit of Provisional Application No. 62/794,387, filed Jan. 18, 2019, which is incorporated herein by reference in its entirety for all purposes.

### FIELD

The disclosure generally relates to implantable medical devices. More specifically, the disclosure is generally directed toward implantable medical devices that include absorbable or bio-degradable filaments.

### BACKGROUND

Medical stents are generally known. One use for medical stents is to support a body lumen, such as a blood vessel, which has contracted in diameter through, for example, the effects of lesions called atheroma or the occurrence of cancerous tumors. Atheroma refers to lesions within arteries that include plaque accumulations that can obstruct blood flow through the vessel. Over time, the plaque can increase in size and thickness and can eventually lead to clinically significant narrowing of the artery, or even complete occlusion. When expanded against the body lumen, which has contracted in diameter, the medical stents provide a tube-like support structure inside the body lumen. At times, stents are lined or covered with thin biocompatible materials. These are called stent grafts and can be used for the endovascular repair of aneurysms. Stents typically are tubular, and are expandable or self-expand from a relatively small diameter to a larger diameter. Stents and stent grafts have also found utility in veins and arteries, and also in bronchial, tracheal, urinary and gastrointestinal applications. Stents may also be used to form an occluder for closure of a tissue opening (e.g., Patent foramen ovale (PFO) or atrial septal defects (ASD)), vascular closure devices, or other similar devices.

### SUMMARY

According to one example (“Example 1”), a medical device includes: a filament; and a membrane arranged about the filament and configured to contain fragments of the filament and maintain structure of the membrane in response to the fracture or degradation of the filament.

According to another example (“Example 2”), further to the medical device of Example 1, the filament is absorbable and configured to degrade over time.

According to another example (“Example 3”), further to the medical device of Example 2, the membrane is configured to contain fragments of the filament during degradation.

According to another example (“Example 4”), further to the medical device of any one of Examples 1-3, the membrane is configured to promote tissue ingrowth, tissue attachment or tissue encapsulation.

According to another example (“Example 5”), further to the medical device of any one of Examples 1-3, the membrane is configured to prevent tissue ingrowth.

According to another example (“Example 6”), further to the medical device of any one of Examples 1-5, the membrane is configured to enhance tensile strength of the filament.

According to another example (“Example 7”), further to the medical device of any one of Examples 1-6, the apparatus also includes an additional membrane layer arranged about the membrane having different material properties than the membrane.

According to another example (“Example 8”), further to the medical device of any one of Examples 1-7, the filament includes a cross-section that is at least one of uneven, jagged, star-like, and polygonal.

According to one example (“Example 9”), a stent includes a plurality of filaments configured to form a scaffold; and a plurality of membranes arranged about each of the plurality of filaments and configured to contain fragments of the plurality of filaments and maintain structure of the scaffold in response to the fracture or degradation of the plurality of filaments.

According to another example (“Example 10”), further to the stent of Example 9, the plurality of filaments are braided to form the scaffold and the plurality of filaments are absorbable and configured to degrade over time.

According to another example (“Example 11”), further to the stent of Example 10, the plurality of membranes are configured to reduce friction between the plurality of filaments.

According to another example (“Example 12”), further to the stent of Example 10, at least at portion of the plurality of membranes is radiopaque.

According to another example (“Example 13”), further to the stent of Example 10, at least at portion of the plurality of membranes includes a drug drug-eluting layer.

According to another example (“Example 14”), further to the stent of Example, the scaffold includes non-absorbable filaments configured to remain in situ after degradation of the plurality of filaments.

According to one example (“Example 15”), an implantable medical device includes a structural element formed by one or more absorbable filaments, the one or more absorbable filaments being configured to degrade over time into a plurality of fragments following implantation, the plurality of fragments including one or more fragments of a first minimum size; and a sheath element at least partially covering the structural element, the sheath element including a membrane and being configured to capture and retain the one or more fragments of the first minimum size during degradation of the of the one or more absorbable filaments.

According to one example (“Example 16”), a method of manufacturing an implantable medical device includes arranging a plurality of membranes about each of a plurality of absorbable filaments to form covered absorbable filaments, the plurality of membranes being configured to contain fragments of the plurality of absorbable filaments in response to the fracture or degradation of the plurality of filaments; and arranging the covered absorbable filaments together to form a scaffold.

According to another example (“Example 17”), further to the method of Example 16, arranging the covered absorbable filaments together includes braiding the covered absorbable filaments to form the scaffold.

According to one example (“Example 18”), a method of treating an opening in a patient to lessen risk of liberating particulate degradation products and/or reduce adverse events caused by emboli in the vascular system from degradation products includes delivering a scaffold within an opening at a treatment site, wherein the scaffold comprises a plurality of absorbable filament and a plurality of membranes arranged about each of the plurality of filaments and the plurality of membranes are configured to contain fragments of the plurality of absorbable filaments within the plurality of membranes in response to the fracture or degradation of the plurality of filaments.

According to one example (“Example 19”), a method of stabilizing tissue includes arranging a suture to span an opening in the tissue, the suture including a filament and a membrane arranged about the filament and configured to contain fragments of the filament and maintain structure of the membrane in response to the fracture or degradation of the filament; and structuring supporting the tissue to promote healing.

According to another example (“Example 20”), further to the method of Example 19, the filament includes at least one of a textured, non-linear, a patterned exterior surface.

According to another example (“Example 21”), further to the method of Example 19, the filament includes an eyelet arranged at one or both ends and further including wrapping the suture about itself through the eyelet.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of an example filament, in accordance with an embodiment; and

FIG. 2 is an illustration of another example filament, in accordance with an embodiment;

FIG. 3 is an illustration of an example implantable medical device, in accordance with an embodiment;

FIG. 4 is an example braiding of an example implantable medical device, in accordance with an embodiment;

FIGS. 5A-C are illustrations of example filament cross-sections, in accordance with an embodiment;

FIG. 6 is an example filament and example membrane, in accordance with an embodiment;

FIG. 7 is another example filament and example membrane, in accordance with an embodiment;

FIG. 8 is an example filament used as a suture, in accordance with an embodiment;

FIGS. 9A-C are illustrations of example filaments, in accordance with an embodiment;

FIG. 10A is an example filament used as a suture in a first configuration, in accordance with an embodiment;

FIG. 10B is the filament, shown in FIG. 10A, in a second configuration, in accordance with an embodiment; and

FIG. 11 shows an example stabilization of fragments of an example filament, in accordance with an embodiment.

### DETAILED DESCRIPTION

### Definitions and Terminology

Persons skilled in the art will readily appreciate that various aspects of the present disclosure can be realized by any number of methods and apparatuses configured to perform the intended functions. It should also be noted that the accompanying drawing referred to herein are not necessarily drawn to scale, but may be exaggerated to illustrate various aspects of the present disclosure, and in that regard, the drawing figures should not be construed as limiting.

This disclosure is not meant to be read in a restrictive manner. For example, the terminology used in the application should be read broadly in the context of the meaning those in the field would attribute such terminology.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Various aspects of the present disclosure are directed toward absorbable filaments (e.g., bio-degradable and/or bio-corrodible) that includes one or more membrane layers. The membrane may remain during and after degradation of the absorbable filament. The membrane may contain pieces or fragments (or particles) of the absorbable filament during and after the degradation period. The membrane may lessen the chance of emboli liberation.

Various aspects of the present disclosure are directed toward medical devices having one or more absorbable filaments that are arranged to form the medical device. The absorbable filaments (which may be struts, a fiber, braided, woven fibers, combined fibers, or other structural elements) may degrade or dissolve through one or more varieties of chemical and/or biological based mechanisms that result in a tissue response suitable for the intended implant application. A membrane or sheath may be arranged with or attached to at least a portion of the one or more absorbable filaments. The membrane may be configured to structurally enhance and/or maintain integrity of the absorbable filaments during degradation or fracture. The membrane is engineered to allow the degradation process, yet will not allow the degradation products to pass until they degrade to a size that allows them to pass through the pores in the membrane. The medical devices may include, for example, a stent or stent-graft or other similar devices. In certain instances, the absorbable filaments are configured to structurally enhance or support the space (e.g., a vessel) into which the medical device is implanted.

In certain instances, the absorbable filaments degrade while the membrane facilitates healthy tissue ingrowth or regrowth. This tissue attachment ensures fixation within the anatomy such that the structure provided by the absorbable filaments may become unnecessary. In addition, the membrane may fully encapsulate and provides a porous jacketed material around the filament or filaments. The membrane surrounding a filament may include a tensile strength and toughness to provide ongoing structural integrity while allowing degradation and fluid or moisture exchange to occur thru the open porosity of the membrane to the filament.

Absorbable herein refers to materials capable of being absorbed by the body, be it directly through dissolution or indirectly through degradation of the implant into smaller components that are then absorbed. The term absorbable also is used herein cover a variety of alternative terms to that have been historically utilized interchangeably both within and across surgical disciplines (but intermittently with inferred differentiation), Those terms include, for example, absorbable and its derivatives, degradable and its derivatives, biodegradable and its derivatives, resorbable and its derivatives, bioresorbable and its derivatives, and biocorrodible and its derivatives. The term absorbable, as used herein, may encompass multiple degradation mechanisms, which include, but are not limited to, corrosion and ester hydrolysis. Further reference may be made to Appendix X4 of ASTM F2902-16 for additional absorbable-related nomenclature.

In addition, filaments, as discussed herein, may include a monofilament, which can also be described as a single fiber, strand, wire, rod, bead, or other non-rigid elongated substantially cylindrical embodiment with a longitudinal dimension that exceeds that of its cross section by greater than 100x. The monofilament may optionally possess one or more overlay coatings or other surface modifications to provide features that are not inherent to its underlying base structure.

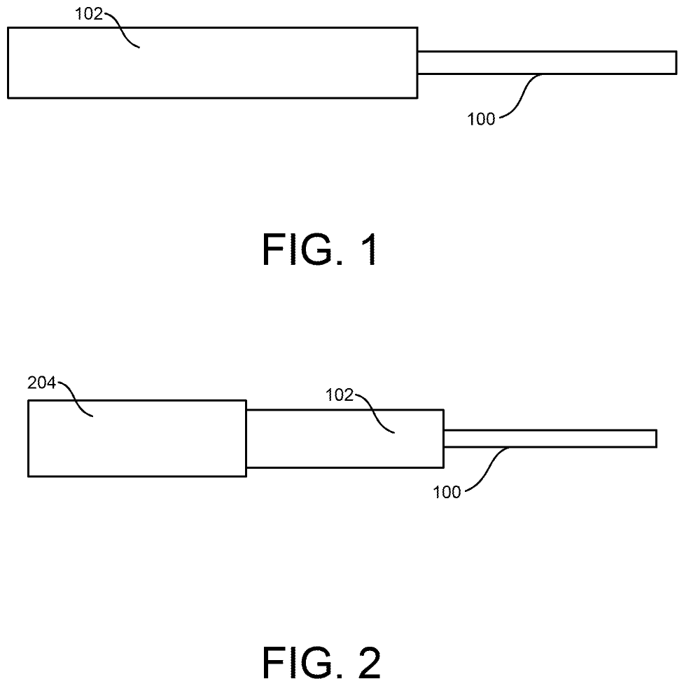

FIG. 1 is an illustration of an example filament 100, in accordance with an embodiment. The filament 100, along with a membrane 102, may form a portion of a medical device, as discussed in further detail below, or be used as with the filament 100 and membrane 102. In certain instances, the membrane 102 is arranged about the filament and configured to initially contain fragments (or particles) of the filament 100 and maintain structure of the membrane 102 in response to the fracture or degradation of the filament 100. The membrane 102 may be coupled or adhered to the filament 100 using a medical adhesive.

In certain instances, the filament 100 is absorbable and configured to degrade over time. The membrane 102 is configured to contain fragments (or particles) of the filament 100 during degradation and absorb into tissue. The membrane 102 may remain in situ after the filament 100, providing a stronger framework than the membrane 102 without the filament 100, has been degraded. The filament 100 may be a structural component that provides a temporary framework to tissue, for example. The temporary framework provided by the filament 100 prior to degradation may facilitate strengthening of the tissue, regrowth of tissue, or growth of healthy tissue. The membrane 102 remains within the patient and provides structure without a metallic framework remaining as would occur with a non-degradable implantable device. In certain instances, the filament 100, acting as a temporary frame structure, is configured to provide enough outward force and/or pressure to allow the membrane 102 to buttress up against the tissue to maintain contact during an initial time period (e.g., 30-60 days) in vivo. This may allow tissue ingrowth, tissue attachment or tissue encapsulation to initiate and provide the early critical anchoring of the filament 100 or device formed by multiple filaments 100 to take place within the tissue bed. The goal will be for the tissue ingrowth, tissue attachment or tissue encapsulation to fully encapsulate the membrane 102 (or device) to maintain its intended shape and position while preventing any embolization of the device

In certain instances, the membrane 102 is configured to promote tissue ingrowth, tissue attachment or tissue encapsulation and in other instances, the membrane 102 is configured to prevent tissue ingrowth. These two states may be affected by designing the membrane component to be porous and controlling the pore size. The porosity of the membrane 102 may control the rate at which the filament 100 degrades. The filament 100 and the membrane 102 may be implanted into a patient to enhance or repair unhealthy tissue. Tissue ingrowth into the membrane 102 (or tissue attachment or tissue encapsulation) may facilitate healthy tissue growth and restoration of the structural integrity of tissue. In certain instances, the filament 100 being degradable allows for initial strengthening of the unhealthy tissue with the generally more bio-compatible membrane 102 remaining in place as opposed to a metallic or semi-metallic filament. In some instances, there may be regions within the same device that have differing needs, therefore combinations of porosity of the membrane 102 may be used (within the same device) to both promote and prohibit tissue ingrowth.

In certain instances, the membrane 102 may be configured to enhance tensile strength of the filament 100. In some cases, the membrane itself will have a stronger tensile strength than the filament it is applied to. This thin, yet strong covering benefits the manufacturing process. The filament is now strong enough to be machine-woven or braided. Membrane 102 may be configured to contain the byproducts of the degradation process for a period of time. The membrane 102 may contain pieces or fragments of the absorbable filament 102 that have been degraded, which may reduce the chance for emboli liberation that could result from fragments of the absorbable filament 102 being released into a patient's bloodstream. The membrane 102 may contain or restrain the products until their physical or chemical dimensions are reduced to a size that allows them to pass through the pores and/or the resulting membrane/tissue composite. In certain instances, the membrane 102 may be configured to maintain fragments from moving away from the treatment site prior to being reduced to sizes sufficiently small that can they can be benignly absorbed by the patient.

In certain instances, the membranes 102 may be absorbable or partially absorbable. The membranes 102, if absorbable, may have an equal or shorter longevity than the absorbable filaments 100. This membranes 102 may enhance/augment tissue coverage over the underlying absorbable filaments 100. This membranes 102 may effectively restrain or contain migration of fragments or particulates that may emanate from the absorbable filaments 100 during degradation or fracture of the absorbable filaments 100. Similar to non-absorbable membranes, the membranes 102 being degradable may allow for tissue attachment and/or ingrowth that stabilizes the overlying tissue so it can contain or substantially restrain migration of fragments and particulate matter emanating from degrading filaments. A porous absorbable membranes 102 that may retain strength and/or ability to provide stable and reinforced overlying tissue for a duration longer than that of the degrading filaments 100 is preferred

FIG. 2 is an illustration of another example filament 100, in accordance with an embodiment. The filament 100 may include a first membrane 102 and a second membrane 204. In certain instances, the membrane 102 is arranged about the filament and configured to contain fragments of the filament 100 and maintain structure of the membrane 102 in response to the fracture or degradation of the filament 100.

In certain instances, the filament 100 is absorbable and configured to degrade over time. The membrane 102 is configured to contain fragments of the filament 100 during degradation .The second membrane 204 is an additional membrane arranged about the (first) membrane 102 having different material properties than the membrane.

One or both of the membranes 102, 204 may remain in situ after the filament 100, providing a stronger framework than the membranes 102, 204 alone, have been degraded. The filament 100 may be a structural component that provides a temporary framework to tissue, for example. The temporary framework provided by the filament 100 prior to degradation may facilitate strengthening of the tissue, regrowth of tissue, or growth of healthy tissue. In certain instances, one of the membranes 102, 204 may be degraded as well as the filament 100. The membranes 102, 204 may facilitate degradation of the filament 100 at different rates than if only one of the membranes 102, 204. In addition, one of the membranes 102, 204 may be a drug-eluting layer.

For instance, filament 100 may be an absorbable metal (such as magnesium), membrane 102 may be a degradable polymer, including a degradable polymer containing a therapeutic agent. Membrane 204 may be a non-degradable polymer (such as ePTFE). This device may also provide radiopacity and initial strength due to the metal framework of the filament 100, if the filament 100 is formed of a metallic degradable material or the device may include radiopacity if the membrane 204 is imbibed with radiopaque material. The two coverings may delay the degradation of the metal by inhibiting the bio-corrosion process. The membrane 102 will begin to degrade and release the therapeutic agent. The membrane 204 will have an engineered porosity that controls therapeutic drug release, contains degradation products until they are degraded to a size that allows them to pass through the pores and, allows for tissue ingrowth, tissue attachment or tissue encapsulation. In certain instances, the filaments 100 may be include a hydrophilically treated film for improved wet-out and chemical diffusion during degradation.

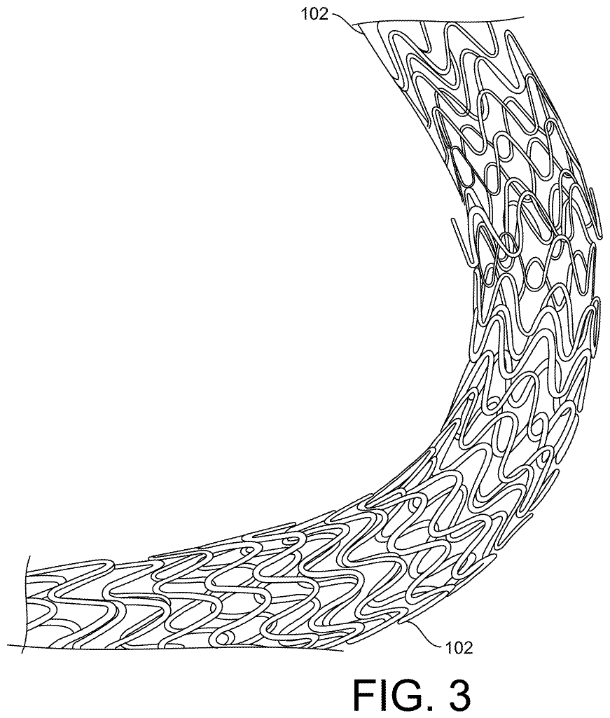

FIG. 3 is an illustration of an example implantable medical device 300, in accordance with an embodiment. The implantable medical device 300 may include one or more absorbable filaments 100. The absorbable filaments 100 may be bio-corrodible, bio-degradable, or both (e.g., a combination of) bio-corrodible and bio-degradable. In addition, the absorbable filaments 100 may include a sheath element at least partially covering the one or more absorbable filaments 100. The one or more absorbable filaments 100 may form a structural element, and therefore, the sheath element may at least partially cover the structural element as shown in FIG. 3. In certain instances, the sheath element covers the entirety of the structural element. The sheath element may include a membrane 102.

In certain instances and as is shown in FIG. 3, each of the one or more absorbable filaments 100 are individually covered by the membrane 102. In addition, the absorbable filaments 100 may be configured to degrade over time into a plurality of fragments following implantation. The plurality of fragments may include one or more fragments of a first minimum size. The membrane 102 is configured to capture and retain the one or more fragments of the first minimum size during degradation of the of the one or more absorbable filaments 100.

As noted above, the absorbable (e.g., bio-degradable, bio-corrodible) filaments 100 are configured to structurally enhance or support the space (e.g., a vessel) into which the medical device 300 is implanted. In certain instances, the absorbable filaments 100 degrade while the membrane 102 (e.g., membrane) facilitates healthy tissue ingrowth or regrowth, tissue attachment or tissue encapsulation such that the structure provided by the absorbable filaments 100 may become unnecessary.

The absorbable filaments 100 forming the medical device 300 may be self-expanding and/or plastically deformable. The absorbable filaments 100 and sheath element may be less abrasive to tissue as compared to a medical device with a metallic based structural element. In this regard, the absorbable filaments 100 may be conformable to structures where involuntary motion is present (e.g., pulsating blood vessels, beating heart, inflating lungs). In certain instances, the absorbable filaments 100 may absorb the involuntary motion.

In addition, the sheath element may include at least a portion of the membrane 102 with a microstructure (e.g., ePTFE) that promotes tissue ingrowth, tissue attachment or tissue encapsulation. In certain instances, the tissue ingrowth may occur in each of the membrane microstructure and the macrostructure of the absorbable filaments 100. In certain instances, the medical device 300 may be occlusive with no covering (e.g., hydrophobic ePTFE).

As noted above, each of the one or more absorbable filaments 100 may be individually covered by the membrane 102. Thus, the medical device 300 may include a plurality of membranes arranged about each of the plurality of filaments 100 or a portion of the plurality of filament(s). The plurality of membranes 102 may be configured to contain fragments of the plurality of filaments 100 and maintain structure of the medical device 300 in response to the fracture or degradation of the plurality of plurality of filaments 100.

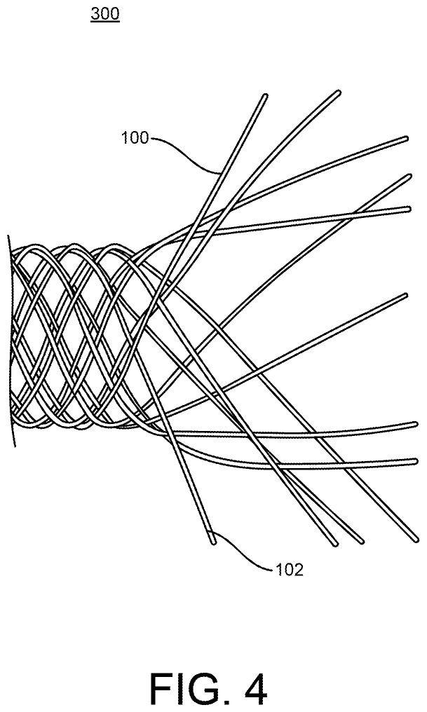

In certain instances, the absorbable filaments 100 may form a braided medical device 300 as described in further detail with reference to FIG. 4. The filaments (e.g., absorbable or non-absorbable) 100 may be braided to form a scaffold and the absorbable filaments 100 are configured to degrade over time. In addition, the membranes 102, 204 may be configured to reduce friction between the plurality of filaments 100. The membrane 102 may be of a material with very low coefficient of friction (e.g., ePTFE). The membrane 102 having a low coefficient of friction will allow the absorbable filaments 100 of a braid to slip past one another. In certain instances, the absorbable filaments 100 and the membrane 102 having a low coefficient of friction (e.g., lower than uncovered) helps in creating a device that compacts and deploys well (and may include a more smooth surface than an uncovered filament 100). As stated earlier, the membrane 102 can add tensile strength and lubricity to the filament, which assists in many of the manufacturing processes (e.g., braiding). In certain instances, braiding provides a stent structure that is flexible and conformable in nature allowing the device 300 to conform naturally to the tissue and anatomy. The braid construct is also balanced with an even number helically wound and interwoven filaments 100 in multiple directions. The balancing of the braid may allow for device 300 to naturally expand into its intended shape by lessening internal bound twisting or bending forces.

In certain instances and as shown, the implantable medical device 300 may be a stent implanted within a patient's vasculature. In other instances, the filaments 100 may be braided into an occluder or other implantable medical device 300 that is to be implanted within a tissue opening or defect of a patient. In either instance, the implantable medical device 300 may form a scaffold that delivered within the opening at a treatment site. The scaffold includes the plurality of membranes 102 arranged about each of a plurality of absorbable filaments 100. The plurality of absorbable filaments 100 may be degraded through through the plurality of membranes 102. During degradation and in response to the fracture or degradation of the plurality of filaments 100, the fragments of the plurality of absorbable filaments 100 are contained within the plurality of membranes 102. After and during degradation of the plurality of filaments 100, the scaffold of the device 300 is formed by the plurality of membranes 102, which remain at the treatment site in the patient. Thus, the scaffold is maintained within the opening after degradation of the plurality of filaments 100.

The membranes 102 facilitate healthy tissue ingrowth or regrowth or tissue attachment or tissue encapsulation. This tissue attachment to the membranes 102 ensures fixation within the anatomy such that the structure provided by the absorbable filaments 100 may become unnecessary. The membranes 102 may possess surface structure may stabilize the absorbable filaments 100 such that fragments of the absorbable filaments 100 are restricted from movement from the treatment site.

In addition, the membranes 102 may fully encapsulate and provides a porous jacketed material around the filament or filaments. The membranes 102 surrounding the filaments 100 may include a tensile strength and toughness to provide ongoing structural integrity while allowing degradation and fluid or moisture exchange to occur thru the open porosity of the membranes 102 to the filaments 100. In certain instances, the filaments 100, acting as a temporary scaffold, are configured to provide enough outward force and/or pressure to allow the membranes 102 to buttress up against the tissue to maintain contact during an initial time period (e.g., 30-60 days) in vivo and maintain a scaffold structure for the tissue after the filaments 100 degrade.

Containing and/or restraining fragments of the plurality of absorbable filaments 100 lessens risk of liberating particulate degradation products as compared to a non-covered absorbable filament. In addition, containing and/or restraining fragments of the plurality of absorbable filaments 100 may reduce the chances of migration and potential adverse events caused by thrombus formation or the generation of emboli in the vascular system.

In certain instances, the device 300 (and other devices discussed herein) may be formed of absorbable and non-absorbable filaments 100. In these instances, some of the scaffold of the device 300 formed by non-absorbable filaments 100 may remain in situ. In instances where the device 300 (and other devices discussed herein) include absorbable and non-absorbable filaments 100, the structural integrity of tissue may be supported in addition to having the membranes 102 remain in vivo by non-absorbable filaments 100 remaining in vivo.

FIG. 4 is an example braiding of an example implantable medical device 300, in accordance with an embodiment. The medical device 300 may include a scaffold of absorbable filaments 100 that are each individually covered with a membrane 102. The plurality of membranes 102 are arranged about each of the plurality of filaments and configured to contain and/or restrain fragments of the plurality of filaments 100 and maintain structure of the scaffold in response to the fracture or degradation of the plurality of plurality of filaments 102.

In certain instances, the filaments 102 may be braided as shown in FIG. 4. The braided medical device 300 may be self-expanding and/or plastically deformable such that the braid conforms to various shapes for various applications. In addition, the membranes 102 may be arranged about the filaments 102 to form covered absorbable filaments 102 prior to being arranged together. In certain instances, the covered absorbable filaments 102 may be braided, interwoven, interlocked, or otherwise arranged together to form the medical device 300.

The membranes 102 may be wrapped about the absorbable filaments 102 in certain instances. Wrapping may act as continuous strength component or member along a braid or filament path allowing for the internal absorbable filaments 100 to be intentionally weakened or broken to see initial fracture points. In addition, the covered absorbable filaments 102 may be shape set to the shape of the medical device 300 after or during braiding. In certain instances, the membrane(s) 102 can provide reinforcement of the filaments 100 using a heat set process and keep the drawn filaments 100 from shrinkage and growing in cross-sectional area. This may allow for higher heat settings to be used and potentially improve crystallization (strength) of the filaments 100 while maintaining smooth and non-distortion of braided construct. The shape set process may also occur using a solvent, and may also occur through other means such as polymeric imbibing through an appropriate fluid or heat setting.

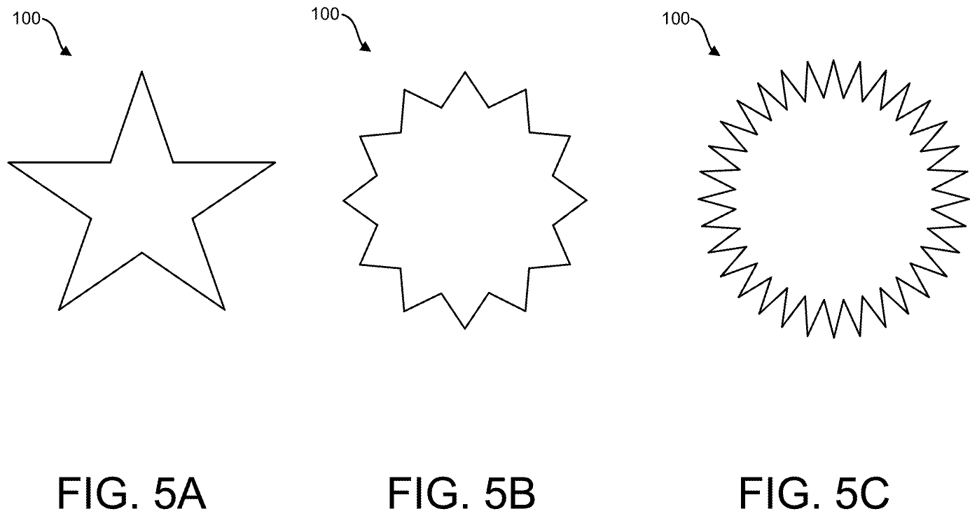

FIGS. 5A-C are illustrations of example filament 100 cross-sections, in accordance with an embodiment. As shown and discussed in detail above, a filament 100 may include a substantially circular cross-section. In other instances and as shown in FIGS. 5A-C, the filament 100 may include a cross-section that is not substantially circular in cross-section.

The filament 100, for example, may be formed or drawn to include a star-like cross-section. The star-like or polygonal cross-section of the filament 100, as shown in FIGS. 5A-C, may increase surface area of the filament 100 as compared to a filament 100 having a substantially circular cross-section. As a result, the degradation profile of the filament 100 may be tailored based on the cross-section of the filament 100. The filament 100, for example, may have a faster degradation profile or rate with a greater surface area. Although the filaments 100 shown in FIGS. 5A-C include specific shapes, the filaments 100 as discussed herein may include uneven, jagged, or patch sides, or include more or less sides than those shown in FIGS. 5A-C (e.g., a triangle, square, pentagon, hexagon). In certain instances, the filaments 100, as discussed herein, may be hollow (e.g., microtubing).

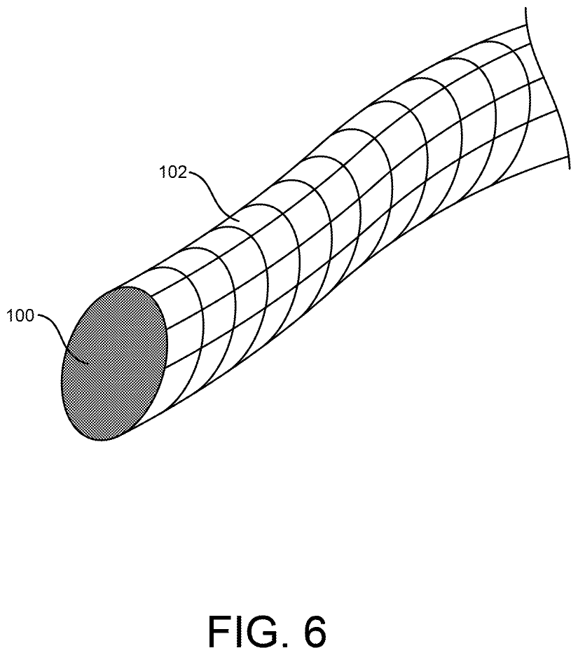

FIG. 6 is an example filament 100 and example membrane 102, in accordance with an embodiment. The filament 100, along with a membrane 102, may form a portion of a medical device, as discussed in further detail below, or be used as with the filament 100 and membrane 102. In certain instances, the membrane 102 is arranged about the filament 100 and configured to initially contain fragments of the filament 100 and maintain structure of the membrane 102 in response to the fracture or degradation of the filament 100. The membrane 102 may be coupled or adhered to the filament 100 using a medical adhesive. The membrane 102 may be non-absorbable and the filament 100 may be absorbable as discussed in detail above.

In certain instances, the membrane 102 may be compressed in one or more directions (e.g., “x” direction). The compression of the membrane 102 may introduce “buckles” or structures that are out-of-plane (i.e., in the “z” direction). Such a process is generally disclosed in U.S. Patent Publication No. 2016/0167291 to Zaggl et al. in which a membrane 102 is applied onto a stretchable substrate in a stretched state such that a reversible adhesion of the membrane 102 on the stretched stretchable substrate occurs.

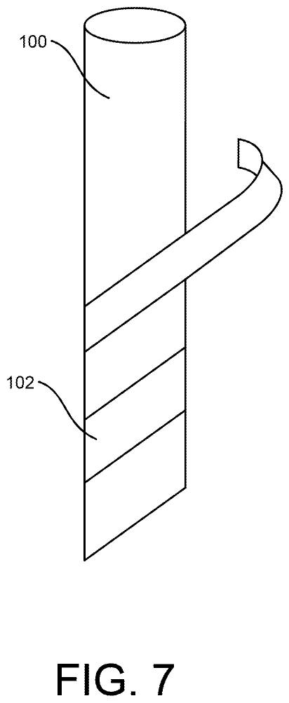

FIG. 7 is another example filament 100 and example membrane 102, in accordance with an embodiment. The filament 100, along with a membrane 102, may form a portion of a medical device, as discussed in further detail below, or be used as with the filament 100 and membrane 102. In certain instances, the membrane 102 is arranged about the filament 100 and configured to initially contain fragments of the filament 100 and maintain structure of the membrane 102 in response to the fracture or degradation of the filament 100. The membrane 102 may be coupled or adhered to the filament 100 using a medical adhesive. The membrane 102 may be non-absorbable and the filament 100 may be absorbable as discussed in detail above.

As shown, the membrane 102 may be wrapped about the filament 100. In certain instances, the membrane 102 is helically wrapped about the filament 100. In these instances, the membrane 102 may partially overlap adjacent windings. The membrane 102 may be adhere to the filament 100 and/or overlapping portions of the adjacent membrane 102 windings. The membrane 102 may be adhered to the filament 100 and/or itself using an adhesive (e.g., fluorinated ethylene propylene (FEP)).

In certain instances, the filament 100 may be set into a desired shape. The shape set filament 100 may be helically wound, woven together into a pattern, or include additional shapes for a desired application (e.g., needless sutures, staple replacements for soft tissue repair).

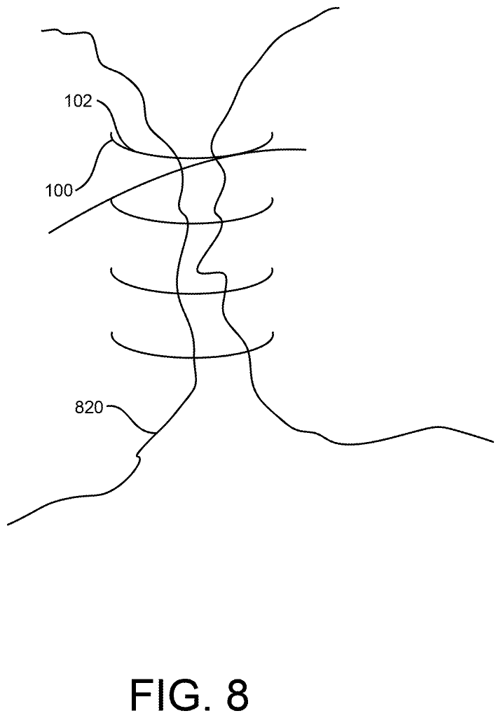

FIG. 8 is an example filament 100 used as a suture, in accordance with an embodiment. As shown in FIG. 8, the filament 100 (wrapped with membrane 102) may be used for tissue 820 repair. As shown, the filament 100 (wrapped with membrane 102) may be used a suture to repair the tissue 820. The filament 100 and membrane 102 may be arranged to span an opening in the tissue 820. The filament 100, prior to degrading, may structurally support the tissue 820 during healing. As the tissue 850 heals, the filament 100 degrades and becomes more compliant. Due to the tissue 850 healing, less structure is needed. The filament 100 degrading in this manner may facilitate faster tissue 820 healing.

In certain instances, the filament 100 may be set into a desired shape. The shape set filament 100 may be helically wound, woven together into a pattern, or include additional shapes for a desired application (e.g., needless sutures, staple replacements for soft tissue repair).

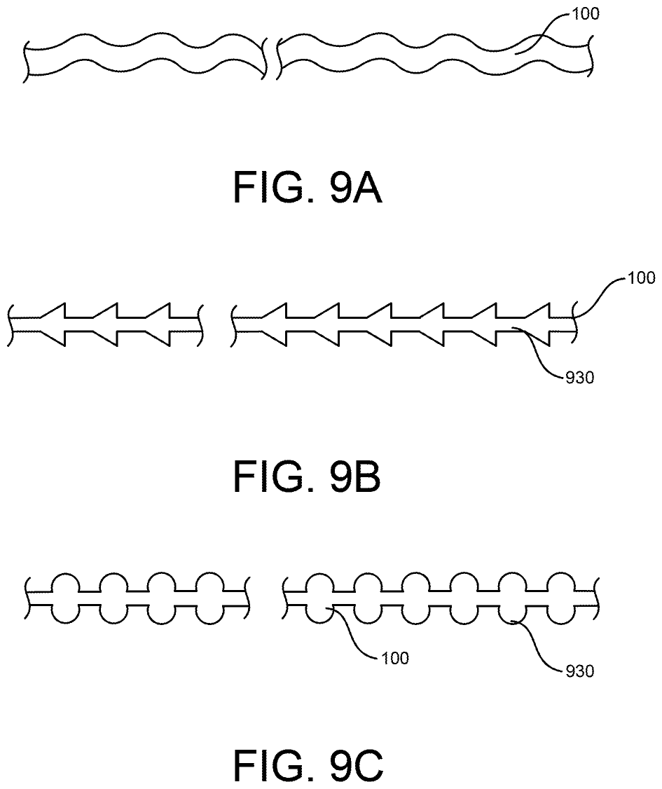

FIGS. 9A-C are illustrations of example filaments 100, in accordance with an embodiment. As shown, the filaments 100 may include textured, non-linear, or patterned exterior surfaces. For example and as shown in FIG. 9A, the filament 100 includes a wave-like structure. In certain instance and as shown in FIGS. 9B-C, the filaments 100 may include one or more protuberances 930. The protuberances 930 may be jagged (e.g., FIG. 9B), semi-circular (FIG. 9C), arranged on one circumferential side of the filaments 100 or on both circumferential sides of the filaments 100 as is shown. The protuberances 930 may facilitate creating knots and knot retention when using the filaments 100, for example, as a suture or thread. The protuberances 930 may enhance friction of the filaments 100 to facilitate tying of knots. In addition, the protuberances 930 may be formed in the filaments 100 and/or a membrane (not shown) arranged about the filaments 100.

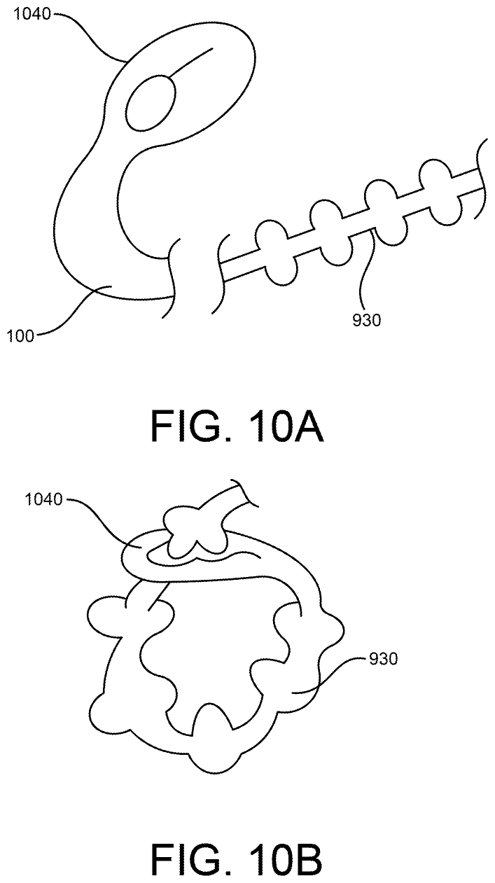

FIGS. 10A-B show an example filament 100 used as a suture in a first configuration (in which the filament 100 is not knotted) and in a second configuration (in which the filament 100 is knotted), in accordance with an embodiment. As shown in FIG. 10A, the filament 100 includes protuberances 930. The filament 100 may also include an eyelet 1040 arranged at one or both ends of the filament 100. As shown in FIG. 10B, the filament 100 may be wrapped about itself through the eyelet 1040. The protuberances 930 may frictionally engage or catch the eyelet 1040 to facilitate knot formation in the filament 100.

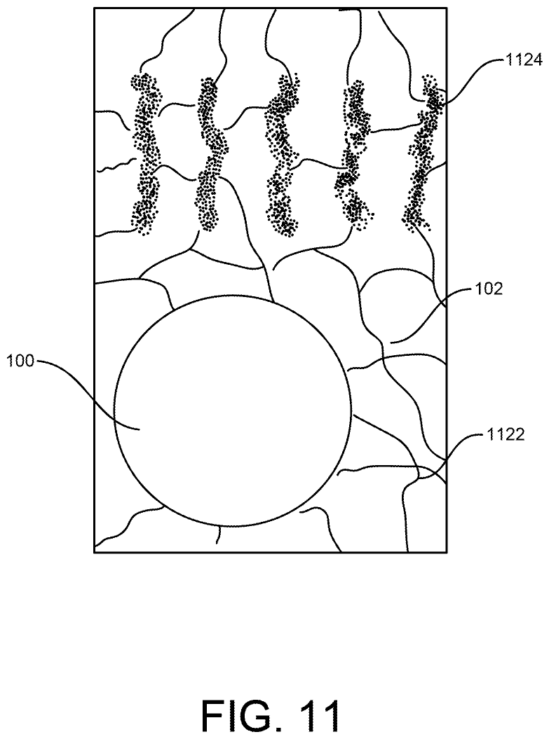

FIG. 11 shows an example stabilization of fragments of an example filament 100, in accordance with an embodiment. As shown in FIG. 11, a membrane 102, may be formed of a scaffold structure (e.g., woven, knitted, non-woven, absorbable, or non-absorbable) components 1122, 1124. The components 1122, 1124 may contain structural components and fragments as the filament 100 degrades. In certain instances, the components 1122, 1124 may also include a porosity to stabilize the fragments and/or particles that may generate from the degrading of the filaments 100 and/or membrane 102. In certain instances, for example, underlying components 1122 may degrade and overlaying components 1124 may stabilize the underlying components 1122. In this manner, the membrane 102 may also degrade and facilitate stabilization as described in detail above.

Upon degradation, the underlying components 1122 may stabilizing the filament 100 and the overlying components 1124. The physical reduction of the overall structural may facilitate degradation of both the filament 100 and portions of the membrane 102 while also integrating the membrane 102 into tissue. The overlying components 1124 may degrade and the underlying components 1122 may integrate into the tissue. The overlying components 1124 degrading (or only the filament 100 degrading with the membrane 102 being non-degradable it its entirety) may facilitate continued tissue coverage and maturation. The overlying components 1124 and the underlying components 1122 may form a continuous membrane 102 or the overlying components 1124 and the underlying components 1122 may be separate structures. In the instances where the overlying components 1124 and the underlying components 1122 are separate structures, the overlying components 1124 may be the membrane 1202 and the underlying components 1122 may be an absorbable layer.

Examples of absorbable filaments include, but are not limited to absorbable metals such as magnesium and magnesium alloys, ferrous materials such as iron, aluminum and aluminum alloys, and other similar materials.

Examples of absorbable polymers that could be used either in the filament or in the membrane component include, but are not limited to, polymers, copolymers (including terpolymers), and blends that may include, in whole or in part, polyester amides, polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) poly(L-lactide-co-glycolide)and copolymeric variants, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolid-caprolactone), poly(dioxanone), poly(ortho esters), poly(trimethylene carbonate), polyphosphazenes, poly(anhydrides), poly(tyrosine carbonates) and derivatives thereof, poly(tyrosine ester) and derivatives thereof, poly(imino carbonates), poly(lactic acid-trimethylene carbonate), poly(glycolic acid-trimethylene carbonate), polyphosphoester, polyphosphoester urethane, poly(amino acids), poly(ethyleneglycol) (PEG), copoly(ether-esters) (e.g. PEO/PLA), polyalkylene oxides such as poly(ethylene oxide), poly(propylene oxide), poly(ether ester), polyalkylene oxalates, poly(aspirin), biomolecules such as collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, chitosan, alginate, or combinations thereof.

Examples of synthetic polymers (which may be used as a membrane) include, but are not limited to, nylon, polyacrylamide, polycarbonate, polyformaldehyde, polymethylmethacrylate, polytetrafluoroethylene, polytrifluorochlorethylene, polyvinylchloride, polyurethane, elastomeric organosilicon polymers, polyethylene, expanded polyethylene, polypropylene, polyurethane, polyglycolic acid, polyesters, polyamides, their mixtures, blends and copolymers are suitable as a membrane material. In one embodiment, said membrane is made from a class of polyesters such as polyethylene terephthalate including DACRON® and MYLAR® and polyaramids such as KEVLAR®, polyfluorocarbons such as polytetrafluoroethylene (PTFE) with and without copolymerized hexafluoropropylene (TEFLON®. or GORE-TEX®.), and porous or nonporous polyurethanes. In certain instances, the membrane comprises expanded fluorocarbon polymers (especially ePTFE) materials. Included in the class of preferred fluoropolymers are polytetrafluoroethylene (PTFE), fluorinated ethylene propylene (FEP), copolymers of tetrafluoroethylene (TFE) and perfluoro(propyl vinyl ether) (PFA), homopolymers of polychlorotrifluoroethylene (PCTFE), and its copolymers with TFE, ethylene-chlorotrifluoroethylene (ECTFE), copolymers of ethylene-tetrafluoroethylene (ETFE), polyvinylidene fluoride (PVDF), and polyvinyfluoride (PVF). Especially preferred, because of its widespread use in vascular prostheses, is ePTFE. In certain instances, the membrane comprises a combination of said materials listed above. In certain instances, the membrane is substantially impermeable to bodily fluids. Said substantially impermeable membrane can be made from materials that are substantially impermeable to bodily fluids or can be constructed from permeable materials treated or manufactured to be substantially impermeable to bodily fluids (e.g. by layering different types of materials described above or known in the art).

Additional examples of membrane materials include, but are not limited to, vinylidinefluoride/hexafluoropropylene hexafluoropropylene (HFP), tetrafluoroethylene (TFE), vinylidenefluoride, 1-hydropentafluoropropylene, perfluoro(methyl vinyl ether), chlorotrifluoroethylene (CTFE), pentafluoropropene, trifluoroethylene, hexafluoroacetone, hexafluoroisobutylene, fluorinated poly(ethylene-co-propylene (FPEP), poly(hexafluoropropene) (PHFP), poly(chlorotrifluoroethylene) (PCTFE), poly(vinylidene fluoride (PVDF), poly(vinylidene fluoride-co-tetrafluoroethylene) (PVDF-TFE), poly(vinylidene fluoride-co-hexafluoropropene) (PVDF-HFP), poly(tetrafluoroethylene-co-hexafluoropropene) (PTFE-HFP), poly(tetrafluoroethylene-co-vinyl alcohol) (PTFE-VAL), poly(tetrafluoroethylene-co-vinyl acetate) (PTFE-VAC), poly(tetrafluoroethylene-co-propene) (PTFEP) poly(hexafluoropropene-co-vinyl alcohol) (PHFP-VAL), poly(ethylene-co-tetrafluoroethylene) (PETFE), poly(ethylene-co-hexafluoropropene) (PEHFP), poly(vinylidene fluoride-co-chlorotrifluoroe-thylene) (PVDF-CTFE), and combinations thereof, and additional polymers and copolymers described in U.S. Publication 2004/0063805, incorporated by reference herein in its entirety for all purposes. Additional polyfluorocopolymers include tetrafluoroethylene (TFE)/perfluoroalkylvinylether (PAVE). PAVE can be perfluoromethylvinylether (PMVE), perfluoroethylvinylether (PEVE), or perfluoropropylvinylether (PPVE). Other polymers and copolymers include, polylactide, polycaprolacton-glycolide, polyorthoesters, polyanhydrides; poly-aminoacids; polysaccharides; polyphosphazenes; poly(ether-ester) copolymers, e.g., PEO-PLLA, or blends thereof, polydimethyl-siolxane; poly(ethylene-vingylacetate); acrylate based polymers or copolymers, e.g., poly(hydroxyethyl methylmethacrylate, polyvinyl pyrrolidinone; fluorinated polymers such as polytetrafluoroethylene; cellulose esters and any polymer and co polymers.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A medical device comprising:
a filament; and
a membrane arranged about the filament and configured to contain fragments of the filament and maintain structure of the membrane in response to the fracture or degradation of the filament.

2. The medical device of claim 1, wherein the filament is absorbable and configured to degrade over time.

3. The medical device of claim 2, wherein the membrane is configured to contain fragments of the filament during degradation.

4. The medical device of claim 1, wherein the membrane is configured to promote tissue ingrowth, tissue attachment, or tissue encapsulation.

5. The medical device of claim 1, wherein the membrane is configured to prevent tissue ingrowth.

6. The medical device of claim 1, wherein the membrane is configured to enhance tensile strength of the filament.

7. The medical device of claim 1, further comprising an additional membrane layer arranged about the membrane having different material properties than the membrane.

8. The medical device of claim 1, wherein the filament includes a cross-section that is at least one of uneven, jagged, star-like, and polygonal.

9. A stent comprising:
a plurality of filaments configured to form a scaffold; and
a plurality of membranes arranged about each of the plurality of filaments and configured to contain fragments of the plurality of filaments and maintain structure of the scaffold in response to the fracture or degradation of the plurality of filaments.

10. The stent of claim 9, wherein the plurality of filaments are braided to form the scaffold and the plurality of filaments are absorbable and configured to degrade over time.

11. The stent of claim 10, wherein the plurality of membranes are configured to reduce friction between the plurality of filaments.

12. The stent of claim 10, wherein at least at portion of the plurality of membranes is radiopaque.

13. The stent of claim 10, wherein at least at portion of the plurality of membranes includes a drug drug-eluting layer.

14. The stent of claim 10, wherein the scaffold includes non-absorbable filaments configured to remain in situ after degradation of the plurality of filaments.

15. An implantable medical device comprising:
a structural element formed by one or more absorbable filaments, the one or more absorbable filaments being configured to degrade over time into a plurality of fragments following implantation, the plurality of fragments including one or more fragments of a first minimum size; and
a sheath element at least partially covering the structural element, the sheath element including a membrane and being configured to capture and retain the one or more fragments of the first minimum size during degradation of the of the one or more absorbable filaments.

16. A method of manufacturing an implantable medical device, the method comprising:
arranging a plurality of membranes about each of a plurality of absorbable filaments to form covered absorbable filaments, the plurality of membranes being configured to contain fragments of the plurality of absorbable filaments in response to the fracture or degradation of the plurality of filaments; and
arranging the covered absorbable filaments together to form a scaffold.

17. The method of claim 16, wherein arranging the covered absorbable filaments together includes braiding the covered absorbable filaments to form the scaffold.

18. A method of treating an opening in a patient to lessen risk of liberating particulate degradation products and/or reduce adverse events caused by emboli in the vascular system from degradation products, the method comprising:
delivering a scaffold within an opening at a treatment site, wherein the scaffold comprises a plurality of absorbable filament and a plurality of membranes arranged about each of the plurality of filaments and the plurality of membranes are configured to contain fragments of the plurality of absorbable filaments within the plurality of membranes in response to the fracture or degradation of the plurality of filaments.

19. A method of stabilizing tissue, the method comprising:
arranging a suture to span an opening in the tissue, the suture including a filament and a membrane arranged about the filament and configured to contain fragments of the filament and maintain structure of the membrane in response to the fracture or degradation of the filament; and
structuring supporting the tissue to promote healing.

20. The method of claim 19, wherein the filament includes at least one of a textured, non-linear, a patterned exterior surface.

21. The method of claim 19, wherein the filament includes an eyelet arranged at one or both ends and further including wrapping the suture about itself through the eyelet.
